(19) United States
(12) Patent Application Publication
(10) Pub. No.: US 2002/0019660 A1
(43) Pub. Date: Feb. 14, 2002

(54) Methods and apparatus for a curved stent

(76) Inventors: Marc Gianotti, Wiesendangen (CH); Kenneth J. Michlitsch, Schaffhausen (CH); Suk-Woo Ha, Marthalen (CH); Randolf von Oepen, Tuebingen (DE); Gerd Seibold, Ammerbuch (DE)

Correspondence Address: FISH & NEAVE, 1251 AVENUE OF THE AMERICAS, 50TH FLOOR, NEW YORK, NY, 10020-1105, US

(21) Appl. No.: 09/916,394

(22) Filed: Jul. 26, 2001

Related U.S. Application Data
(63) Continuation-in-part of application No. 09/742,144, filed on Dec. 19, 2000.
Application No. 09/742,144 is a continuation-in-part of application No. 09/582,318, filed on Jun. 23, 2000.

(30) Foreign Application Priority Data
Sep. 5, 1998 (DE) DE 198 40 645.2
Sep. 2, 1999 (EP) PCT/EP99/06456

(51) Int. Cl. A61F 2/06

(52) U.S. Cl. 623/1.15

(57) ABSTRACT

The present invention provides a stent comprising a tubular flexible body having a wall with a web structure that is expandable from a contracted delivery configuration to deployed configuration. The web structure comprises a plurality of neighboring, interconnected, web patterns, each web pattern composed of adjoining webs. Each adjoining web comprises a central section interposed between two lateral sections, forming concave or convex configurations. Embodiments of the present invention comprising curvature for tracking tortuous anatomy and reducing localized restoring forces are provided. Methods of using stents in accordance with the present invention are also provided.

### REFERENCE TO RELATED APPLICATIONS

The present application is a continuation-in-part of U.S. patent application Ser. No. 09/742,144, filed Dec. 19, 2000, which is a continuation-in-part of U.S. patent application Ser. No. 09/582,318, filed Jun. 23, 2000, which claims the benefit of the filing date of International Application PCT/EP99/06456, filed Sep. 2, 1999, which claims priority from German application 19840645.2, filed Sep. 5, 1998.

### FIELD OF THE INVENTION

The present invention relates to stents. More particularly, the present invention relates to stents having curvature, and that preferably have web structures configured to expand from contracted delivery configurations to expanded deployed configurations.

### BACKGROUND OF THE INVENTION

Various stent designs are known in the art. These stents form vascular prostheses fabricated from biocompatible materials. Stents are typically used to expand and maintain patency of hollow vessels, such as blood vessels or other body orifices. To this end, the stent is often placed into a hollow vessel of a patient's body in a contracted delivery configuration and is subsequently expanded by suitable means, such as by a balloon catheter or through self-expansion, to a deployed configuration.

A stent often comprises a stent body that is expandable from the contracted to the deployed configuration. A common drawback of such a stent is that the stent decreases in length, or foreshortens, along its longitudinal axis as it expands. Such shortening is undesirable because, in the deployed configuration, the stent may not span the entire area inside a vessel or orifice that requires expansion and/or support. Additionally, when implanted in tortuous anatomy, prior art stents may apply hazardous localized restoring forces to the vessels or orifices.

It therefore would be desirable to provide a stent that experiences reduced foreshortening during deployment.

It also would be desirable to provide a stent that is flexible, even in the contracted delivery configuration.

It would be desirable to provide a stent having radial stiffness in the expanded deployed configuration sufficient to maintain vessel patency in a stenosed vessel.

It would be desirable to provide a stent having curvature adapted to reduce localized restoring forces.

### SUMMARY OF THE INVENTION

In view of the foregoing, it is an object of the present invention to provide a stent that experiences reduced foreshortening during deployment.

It is another object to provide a stent that is flexible, even in the contracted delivery configuration.

It is also an object to provide a stent having radial stiffness in the expanded deployed configuration sufficient to maintain vessel patency in a stenosed vessel.

It is an object to provide a stent having curvature adapted to reduce localized restoring forces. These and other objects of the present invention are accomplished by providing a stent having a tubular body whose wall has a web structure configured to expand from a contracted delivery configuration to an expanded deployed configuration. The web structure comprises a plurality of neighboring web patterns having adjoining webs. Each web has three sections: a central section arranged substantially parallel to the longitudinal axis in the contracted delivery configuration, and two lateral sections coupled to the ends of the central section. The angles between the lateral sections and the central section increase during expansion, thereby reducing or substantially eliminating length decrease of the stent due to expansion, while increasing a radial stiffness of the stent.

Preferably, each of the three sections of each web is substantially straight, the lateral sections preferably define obtuse angles with the central section, and the three sections are arranged relative to one another to form a concave or convex structure. When contracted to its delivery configuration, the webs resemble stacked or nested bowls or plates. This configuration provides a compact delivery profile, as the webs are packed against one another to form web patterns resembling rows of stacked plates.

Neighboring web patterns are preferably connected to one another by connection elements preferably formed as straight sections. In a preferred embodiment, the connection elements extend between adjacent web patterns from the points of interconnection between neighboring webs within a given web pattern. The orientation of connection elements between a pair of neighboring web patterns preferably is the same for all connection elements disposed between the pair. However, the orientation of connection elements alternates between neighboring pairs of neighboring web patterns. Thus, a stent illustratively flattened and viewed as a plane provides an alternating orientation of connection elements between the neighboring pairs: first upwards, then downwards, then upwards, etc.

As will be apparent to one of skill in the art, positioning, distribution density, and thickness of connection elements and adjoining webs may be varied to provide stents exhibiting characteristics tailored to specific applications. Applications may include, for example, use in the coronary or peripheral (e.g. renal) arteries. Positioning, density, and thickness may even vary along the length of an individual stent in order to vary flexibility and radial stiffness characteristics along the length of the stent.

Stents of the present invention preferably are flexible in the delivery configuration. Such flexibility beneficially increases a clinician's ability to guide the stent to a target site within a patient's vessel. Furthermore, stents of the present invention preferably exhibit high radial stiffness in the deployed configuration. Implanted stents therefore are capable of withstanding compressive forces applied by a vessel wall and maintain vessel patency. The web structure described hereinabove provides the desired combination of flexibility in the delivery configuration and radial stiffness in the deployed configuration. The combination further may be achieved, for example, by providing a stent having increased wall thickness in a first portion of the stent and decreased wall thickness with fewer connection elements in an adjacent portion or portions of the stent.

Depending on the material of fabrication, a stent of the present invention may be either self-expanding or expandable by other suitable means, for example, using a balloon catheter. Self-expanding embodiments preferably are fabricated from a superelastic material, such as a nickel-titanium alloy. Regardless of the expansion mechanism used, the beneficial aspects of the present invention are maintained: reduced shortening upon expansion, high radial stiffness, and a high degree of flexibility.

Stents of the present invention may comprise curvature adapted to match the curvature of an implantation site within a patient's body lumen or orifice, for example, adapted to match the curvature of a tortuous blood vessel. Curvature matching is expected to reduce potentially harmful restoring forces that are applied to tortuous anatomy by prior art stents. Such restoring forces may cause local irritation of cells due to force concentration. The forces also may cause vessel kinking, which reduces luminal diameter and blood flow, while increasing blood pressure and turbulence.

Curvature may be imparted to the stents by a variety of techniques, such as by heat treating the stents while they are arranged with the desired curvature, or plastically deforming the stents to a curved configuration with secondary apparatus, e.g. a curved balloon.

Methods of using stents in accordance with the present invention are also provided.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a schematic isometric view illustrating the basic structure of a stent according to the present invention;

FIG. 2 is a schematic view illustrating a web structure of a wall of the stent of FIG. 1 in a contracted delivery configuration;

FIG. 3 is a schematic view illustrating the web structure of the stent of FIG. 1 in an expanded deployed configuration;

FIG. 4 is an enlarged schematic view of the web structure in the delivery configuration;

FIG. 5 is a schematic view of an alternative web structure of the stent of FIG. 1 having transition sections and shown in an as-manufactured configuration;

FIGS. 6A and 6B are, respectively, a schematic view and a detailed view of an alternative embodiment of the web structure of FIG. 5;

FIGS. 7A-7D are, respectively, a schematic view and detailed views of another alternative embodiment of the web structure of the stent of the present invention, and a cross-sectional view of the stent;

FIGS. 8A and 8B are schematic views of further alternative embodiments of the stent of the present application having different interconnection patterns;

FIGS. 9A and 9B are, respectively, a schematic and a detailed view of yet another alternative embodiment of the web structure of FIG. 5;

FIGS. 10A-10D are side views, partially in section, illustrating a method of deploying a balloon expandable stent constructed in accordance with the present invention;

FIG. 11 is a side view of a self-expanding stent of the present invention having a curvature relative to a longitudinal axis of the stent;

FIG. 12 is a side view of the stent of FIG. 11 disposed within a delivery catheter;

FIGS. 13A-13C are side views, partially in section, illustrating a method of deploying the stent of FIG. 11 within tortuous anatomy;

FIG. 14 is a schematic view of an optional intravascular ultrasound image provided for positioning of the stent of FIG. 11; and

FIGS. 15A and 15B are side-views of secondary balloon apparatus for imposing curvature on a balloon-expandable stent of the present invention, shown, respectively, in a collapsed delivery configuration, and in an expanded deployed configuration.

### DETAILED DESCRIPTION OF THE INVENTION

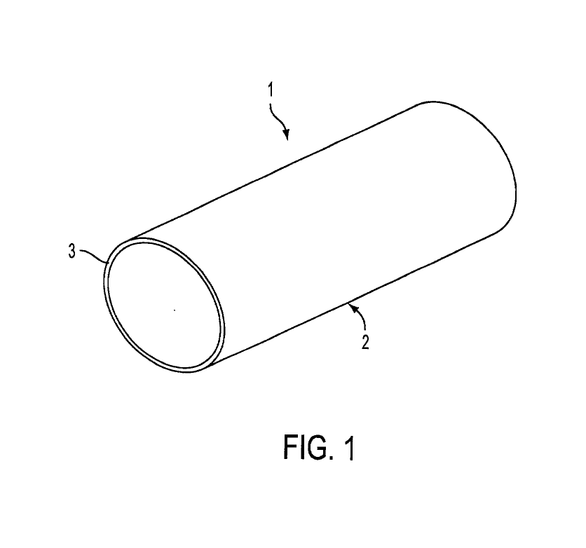

Referring to FIG. 1, stent 1 comprises tubular flexible body 2. Tubular flexible body 2, in turn, comprises wall 3 having a web structure, as described hereinbelow with respect to FIGS. 2-9. Stent 1 and its web structure are expandable from a contracted delivery configuration to an expanded deployed configuration. Depending on the material of fabrication, stent 1 may be either self-expanding or expandable using a balloon catheter or other apparatus. If self-expanding, the web structure is preferably fabricated from a superelastic material, such as a nickel-titanium alloy. Furthermore, stent 1 preferably is fabricated from biocompatible or biodegradable materials. It also may be radiopaque to facilitate delivery, and it may comprise an external coating C that retards thrombus formation or restenosis within a vessel. The coating alternatively may deliver therapeutic agents into the patient's blood stream.

With reference to FIGS. 2-4, a first embodiment of the web structure of stent 1 is described. In FIGS. 2-4, wall 3 of body 2 of stent 1 is shown flattened into a plane for illustrative purposes. FIG. 2 shows web structure 4 in a contracted delivery configuration, with line L indicating the longitudinal axis of the stent. Web structure 4 comprises neighboring web patterns 5 and 6 arranged in alternating, side-by-side fashion. Thus, the web patterns seen in FIG. 2 are arranged in the sequence 5, 6, 5, 6, 5, etc.

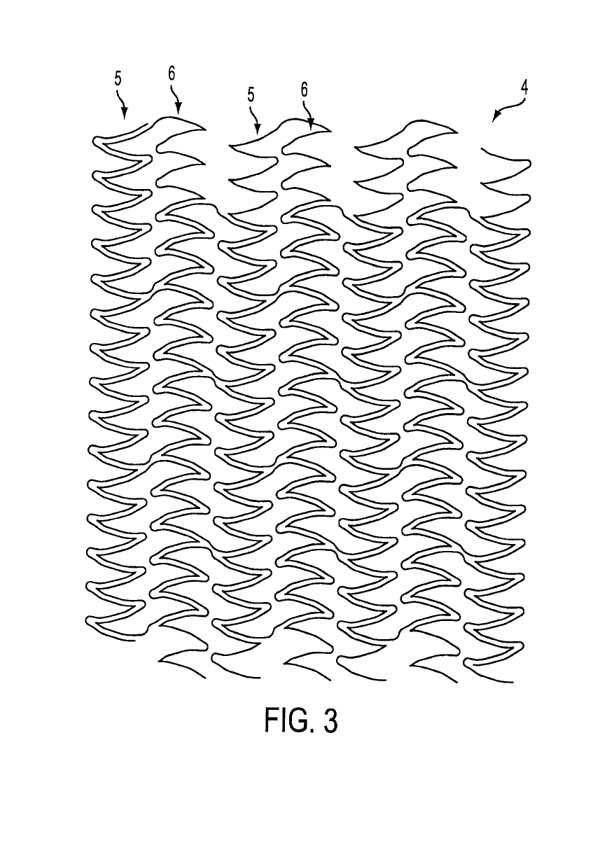

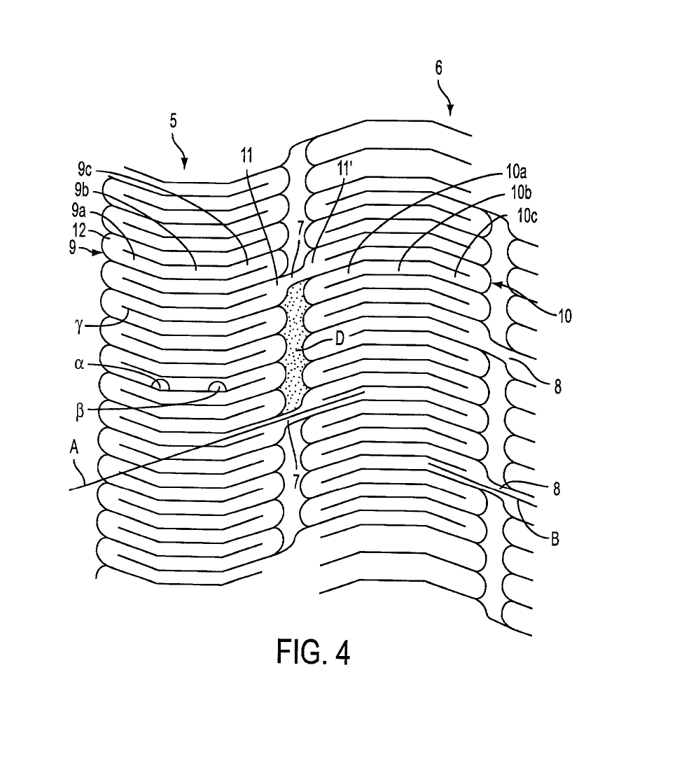

FIG. 2 illustrates that web patterns 5 comprise adjoining webs 9 (concave up in FIG. 2), while web patterns 6 comprise adjoining webs 10 (convex up in FIG. 2). Each of these webs has a concave or convex shape resulting in a stacked plate- or bowl-like appearance when the stent is contracted to its delivery configuration. Webs 9 of web patterns 5 are rotated 180 degrees with respect to webs 10 of web patterns 6, i.e., alternating concave and convex shapes. The structure of webs 9 and 10 is described in greater detail hereinbelow with respect to FIG. 4.

Neighboring web patterns 5 and 6 are interconnected by connection elements 7 and 8. A plurality of connection elements 7 and 8 are provided longitudinally between each pair of web patterns 5 and 6. Multiple connection elements 7 and 8 are disposed in the circumferential direction between adjacent webs 5 and 6. The position, distribution density, and thickness of these pluralities of connection elements may be varied to suit specific applications in accordance with the present invention.

Connection elements 7 and 8 exhibit opposing orientation. However, all connection elements 7 have the same orientation that, as seen in FIG. 2, extends from the left side, bottom, to the right side, top. Likewise, all connection elements 8 have the same orientation that extends from the left side, top, to the right side, bottom. Connection elements 7 and 8 alternate between web patterns 5 and 6, as depicted in FIG. 2.

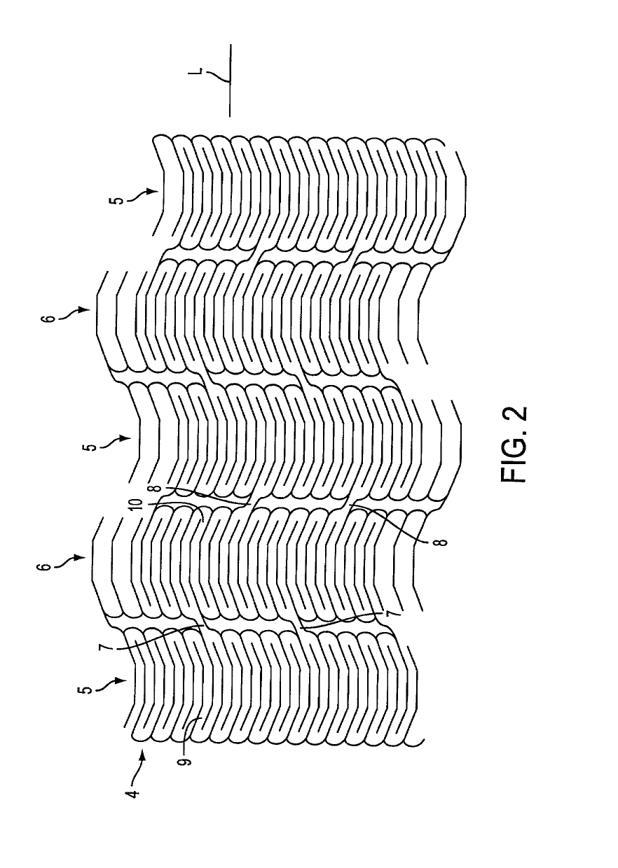

FIG. 3 illustrates the expanded deployed configuration of stent 1, again with reference to a portion of web structure 4. When stent 1 is in the expanded deployed configuration, web structure 4 provides stent 1 with high radial stiffness. This stiffness enables stent 1 to remain in the expanded configuration while, for example, under radial stress. Stent 1 may experience application of radial stress when, for example, implanted into a hollow vessel in the area of a stenosis.

FIG. 4 is an enlarged view of web structure 4 detailing a portion of the web structure disposed in the contracted delivery configuration of FIG. 2. FIG. 4 illustrates that each of webs 9 of web pattern 5 comprises three sections 9a, 9b and 9c, and each of webs 10 of web pattern 6 comprises three sections 10a, 10b and 10c. Preferably, each individual section 9a, 9b, 9c, 10a, 10b and 10c, has a straight configuration.

Each web 9 has a central section 9b connected to lateral sections 9a and 9c, thus forming the previously mentioned bowl- or plate-like configuration. Sections 9a and 9b enclose obtuse angle α. Likewise, central section 9b and lateral section 9c enclose obtuse angle β. Sections 10a-10c of each web 10 of each web pattern 6 are similarly configured, but are rotated 180 degrees with respect to corresponding webs 9. Where two sections 9a or 9c, or 10a or 10c adjoin one another, third angle γ is formed (this angle is zero where the stent is in the fully contracted position, as shown in FIG. 4).

Preferably, central sections 9b and 10b are substantially aligned with the longitudinal axis L of the tubular stent when the stent is in the contracted delivery configuration. The angles between the sections of each web increase in magnitude during expansion to the deployed configuration, except that angle γ, which is initially zero or acute, approaches a right angle after deployment of the stent. This increase provides high radial stiffness with reduced shortening of the stent length during deployment. As will of course be understood by one of ordinary skill, the number of adjoining webs that span a circumference of the stent preferably is selected corresponding to the vessel diameter in which the stent is intended to be implanted.

FIG. 4 illustrates that, with stent 1 disposed in the contracted delivery configuration, webs 9 adjoin each other in an alternating fashion and are each arranged like plates stacked into one another, as are adjoining webs 10. FIG. 4 further illustrates that the configuration of the sections of each web applies to all of the webs, which jointly form web structure 4 of wall 3 of tubular body 2 of stent 1. Webs 9 are interconnected within each web pattern 5 via rounded connection sections 12, of which one connection section 12 is representatively labeled. Webs 10 of each neighboring web pattern 6 are similarly configured.

FIG. 4 also once again demonstrates the arrangement of connection elements 7 and 8. Connection elements 7, between a web pattern 5 and a neighboring web pattern 6, are disposed obliquely relative to the longitudinal axis L of the stent with an orientation A, which is the same for all connection elements 7. Orientation A is illustrated by a straight line that generally extends from the left side, bottom, to the right side, top of FIG. 4. Likewise, the orientation of all connection elements 8 is illustrated by line B that generally extends from the left side, top, to the right side, bottom of FIG. 4. Thus, an alternating A, B, A, B, etc., orientation is obtained over the entirety of web structure 4 for connection elements between neighboring web patterns.

Connection elements 7 and 8 are each configured as a straight section that passes into a connection section 11 of web pattern 5 and into a connection section 11′ of web pattern 6. This is illustratively shown in FIG. 4 with a connection element 7 extending between neighboring connection sections 11 and 11′, respectively. It should be understood that this represents a general case for all connection elements 7 and 8.

Since each web consists of three interconnected sections that form angles α and β with respect to one another, which angles are preferably obtuse in the delivery configuration, expansion to the deployed configuration of FIG. 3 increases the magnitude of angles α and β. This angular increase beneficially provides increased radial stiffness in the expanded configuration. Thus, stent 1 may be flexible in the contracted delivery configuration to facilitate delivery through tortuous anatomy, and also may exhibit sufficient radial stiffness in the expanded configuration to ensure vessel patency, even when deployed in an area of stenosis. The increase in angular magnitude also reduces and may even substantially eliminate length decrease of the stent due to expansion, thereby decreasing a likelihood that stent 1 will not completely span a target site within a patient's vessel post-deployment.

The stent of FIG. 4 is particularly well suited for use as a self-expanding stent when manufactured, for example, from a shape memory alloy such as nickel-titanium. In this case, web patterns 5 and 6 preferably are formed by laser-cutting a tubular member, wherein adjacent webs 9 and 10 are formed using slit-type cuts. Only the areas circumferentially located between connection members 7 and 8 (shaded area D in FIG. 4) require removal of areas of the tubular member. These areas also may be removed from the tubular member using laser-cutting techniques.

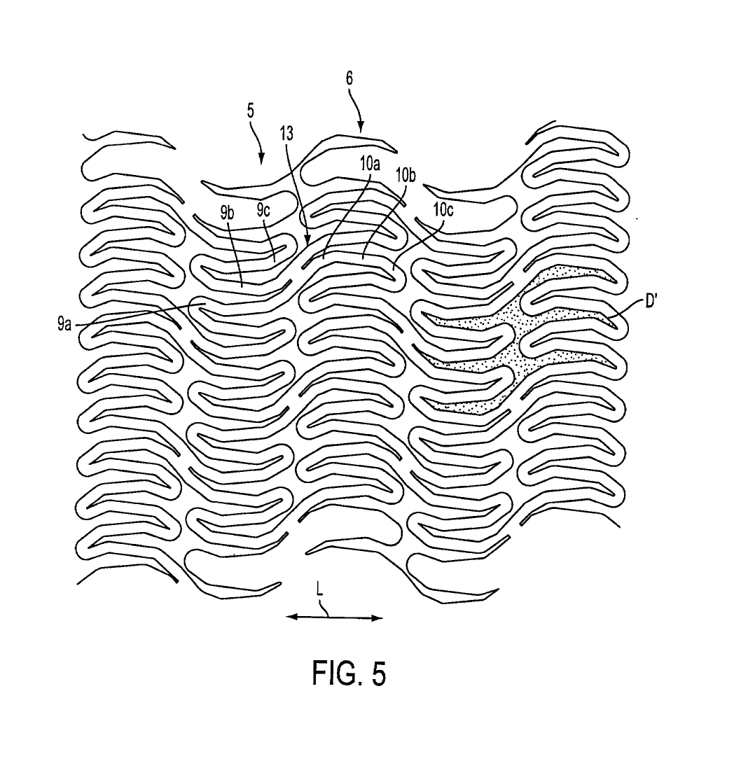

Referring now to FIG. 5, an alternative embodiment of the web structure of stent 1 is described. FIG. 5 shows the alternative web structure in an as-manufactured configuration. The basic pattern of the embodiment of FIG. 5 corresponds to that of the embodiment of FIGS. 2-4. Thus, this alternative embodiment also relates to a stent having a tubular flexible body with a wall having a web structure configured to expand from a contracted delivery configuration to the deployed configuration.

Likewise, the web structure again comprises a plurality of neighboring web patterns, of which two are illustratively labeled in FIG. 5 as web patterns 5 and 6. Web patterns 5 and 6 are again provided with adjoining webs 9 and 10, respectively. Each of webs 9 and 10 is subdivided into three sections, and reference is made to the discussion provided hereinabove, particularly with respect to FIG. 4. As will of course be understood by one of skill in the art, the stent of FIG. 5 will have a smaller diameter when contracted (or crimped) for delivery, and may have a larger diameter than illustrated in FIG. 5 when deployed (or expanded) in a vessel.

The embodiment of FIG. 5 differs from the previous embodiment by the absence of connection elements between web patterns. In FIG. 5, web patterns are interconnected to neighboring web patterns by transition sections 13, as shown by integral transition section 13 disposed between sections 9c and 10c. Symmetric, inverted web patterns are thereby obtained in the region of transition sections 13. To enhance stiffness, transition sections 13 preferably have a width greater than twice the width of webs 9 or 10.

As seen in FIG. 5, every third neighboring pair of webs 9 and 10 is joined by an integral transition section 13. As will be clear to those of skill in the art, the size and spacing of transition sections 13 may be altered in accordance with the principles of the present invention.

An advantage of the web structure of FIG. 5 is that it provides stent 1 with compact construction coupled with a high degree of flexibility in the delivery configuration and high load-bearing capabilities in the deployed configuration. Furthermore, FIG. 5 illustrates that, as with connection elements 7 and 8 of FIG. 4, transition sections 13 have an alternating orientation and are disposed obliquely relative to the longitudinal axis of the stent (shown by reference line L). FIG. 5 also illustrates that, especially in the deployed configuration, an H-like configuration of transition sections 13 with adjoining web sections is obtained.

The stent of FIG. 5 is well suited for use as a balloon-expandable stent, and may be manufactured from stainless steel alloys. Unlike the stent of FIG. 4, which is formed in the contracted delivery configuration, the stent of FIG. 5 preferably is formed in a partially deployed configuration by removing the shaded areas D′ between webs 9 and 10 using laser-cutting or chemical etching techniques. In this case, central sections 9b and 10b are substantially aligned with the longitudinal axis L of the stent when the stent is crimped onto the dilatation balloon of a delivery system.

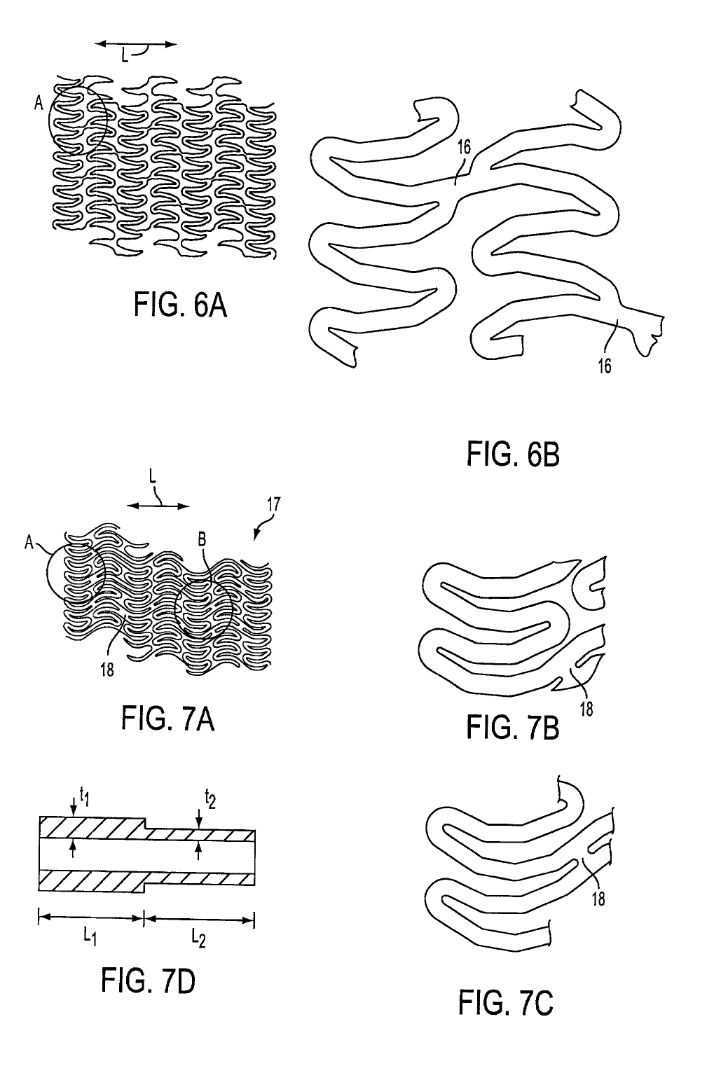

Referring now to FIGS. 6 and 7, alternative embodiments of the web structure of FIG. 5 are described. These web structures differ from the embodiment of FIG. 5 in the spacing of the transition sections. Web structure 15 of FIGS. 6A and 6B provides a spacing of transition sections 16 suited for use in the coronary arteries. FIG. 6A shows the overall arrangement, while FIG. 6B provides a detail view of region A of FIG. 6A. Other arrangements and spacings will be apparent to those of skill in the art and fall within the scope of the present invention.

Web structure 17 of FIGS. 7A-7D provides stent 1 with a variable wall thickness and a distribution density or spacing of transition sections 16 suited for use in the renal arteries. FIG. 7A shows the arrangement of web structure 17 along the length of stent 1, and demonstrates the spacing of transition sections 18. FIGS. 7C and 7D provide detail views of regions A and B. respectively, of FIG. 7A, showing how the spacing and shape of the webs that make up web structure 17 change as stent 1 changes along its length. In particular, as depicted (not to scale) in FIG. 7D, stent 1 has first thickness t₁ for first length L₁ and second thickness t₂ for second length L₂.

The variation in thickness, rigidity and number of struts of the web along the length of the stent of FIGS. 7A-7D facilitates use of the stent in the renal arteries. For example, the thicker region L₁ includes more closely spaced and sturdier struts to provide a high degree of support in the ostial region, while the thinner region L₂ includes fewer and thinner struts to provide greater flexibility to enter the renal arteries. For such intended applications, region L₁ preferably has a length of about 6-8 mm and a nominal thickness t₁ of 0.21 mm, and region L₂ has a length of about 5 mm and a nominal thickness t₂ of about 0.15 mm.

As depicted in FIGS. 7A-7D, the reduction in wall thickness may occur as a step along the exterior of the stent, such as may be obtained by grinding or chemical etching. One of ordinary skill in the art will appreciate, however, that the variation in thickness may occur gradually along the length of the stent, and that the reduction in wall thickness could be achieved by alternatively removing material from the interior surface of the stent, or both the exterior and interior surfaces of the stent.

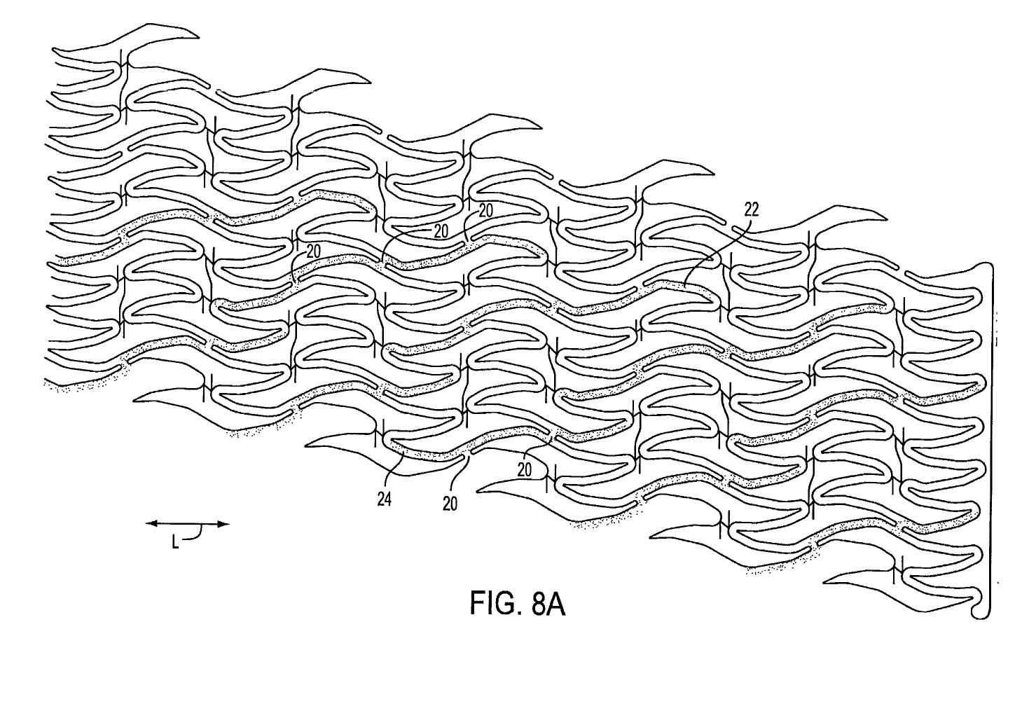

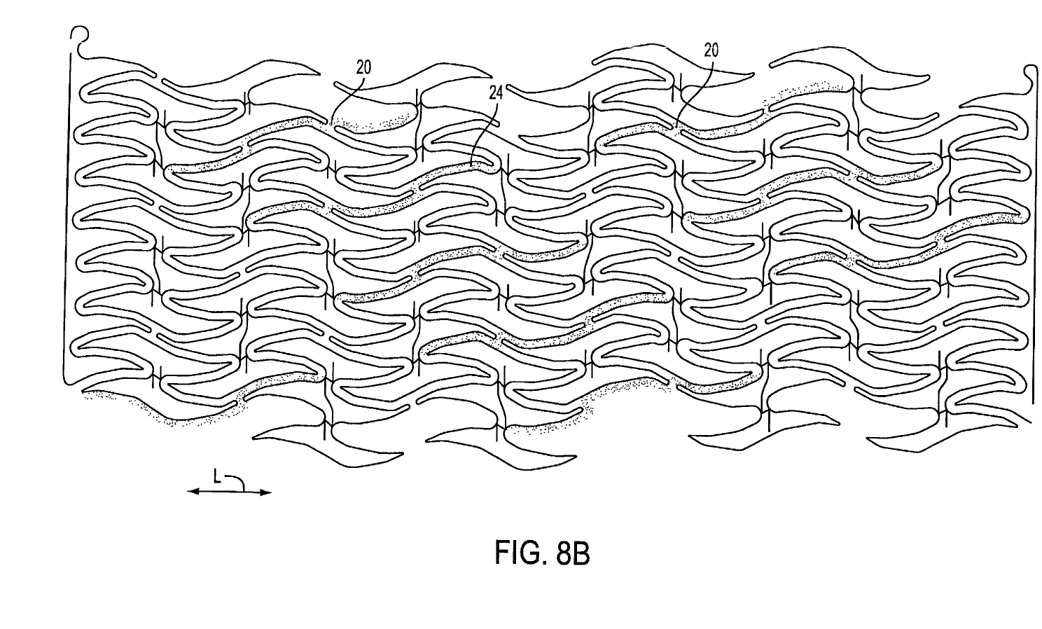

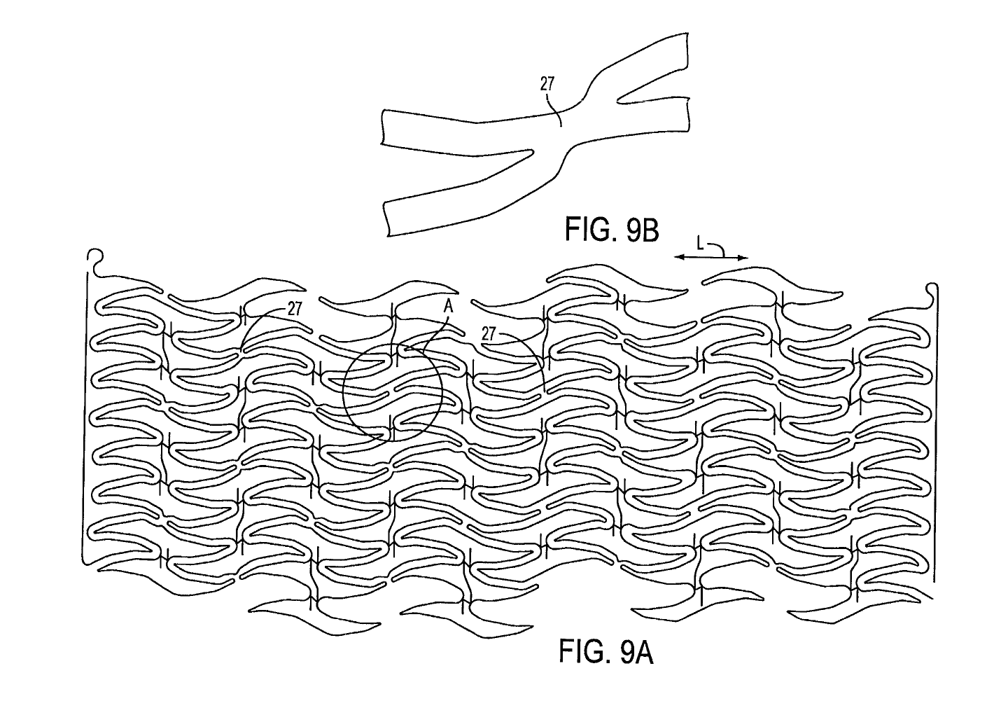

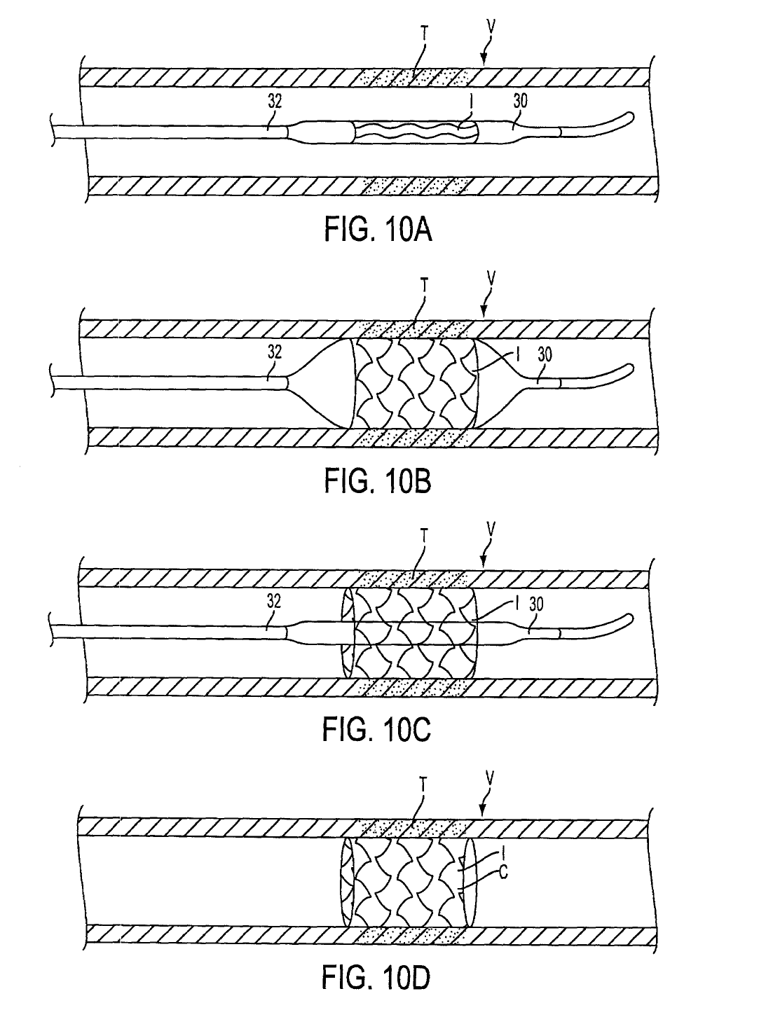

In FIGS. 8A and 8B, additional embodiments of web structures of the present invention, similar to FIG. 5, are described; in which line L indicates the direction of the longitudinal axis of the stent. In FIG. 5, every third neighboring pair of webs is joined by an integral transition section 13, and no set of struts 9a-9c or 10a-10c directly joins two transition sections 13. In the embodiment of FIG. 8A, however, integral transition sections 20 are arranged in a pattern so that the transition sections span either four or three adjacent webs. For example, the portion indicated as 22 in FIG. 8A includes three consecutively joined transition sections, spanning four webs. In the circumferential direction, portion 22 alternates with the portion indicated at 24, which includes two consecutive transition sections, spanning three webs.

By comparison, the web pattern depicted in FIG. 8B includes only portions 24 that repeat around the circumference of the stent, and span only three webs at a time. As will be apparent to one of ordinary skill, other arrangements of integral transition regions 13 may be employed, and may be selected on an empirical basis to provide any desired degree of flexibility and trackability in the contracted delivery configuration, and suitable radial strength in the deployed configuration.

Referring now to FIGS. 9A and 9B, a further alternative embodiment of the stent of FIG. 8B is described, in which the transition sections are formed with reduced thickness. Web structure 26 comprises transition sections 27 disposed between neighboring web patterns. Sections 27 are thinner and comprise less material than transition sections 20 of the embodiment of FIG. 8B, thereby enhancing flexibility without significant reduction in radial stiffness.

Referring now to FIGS. 10A-10D, a method of using a balloon expandable embodiment of stent 1 is provided. Stent 1 is disposed in a contracted delivery configuration over balloon 30 of balloon catheter 32. As seen in FIG. 10A, the distal end of catheter 32 is delivered to a target site T within a patient's vessel V using, for example, well-known percutaneous techniques. Stent 1 or portions of catheter 32 may be radiopaque to facilitate positioning within the vessel. Target site T may, for example, comprise a stenosed region of vessel V at which an angioplasty procedure has been conducted.

In FIG. 10B, balloon 30 is inflated to expand stent 1 to the deployed configuration in which it contacts the wall of vessel V at target site T. Notably, the web pattern of stent 1 described hereinabove minimizes a length decrease of stent 1 during expansion, thereby ensuring that stent 1 covers all of target site T. Balloon 30 is then deflated, as seen in FIG. 10C, and balloon catheter 32 is removed from vessel V, as seen in FIG. 10D.

Stent 1 is left in place within the vessel. Its web structure provides radial stiffness that maintains stent 1 in the expanded configuration and minimizes restenosis. Stent 1 may also comprise external coating C configured to retard restenosis or thrombosis formation around the stent. Coating C may alternatively deliver therapeutic agents into the patient's blood stream.

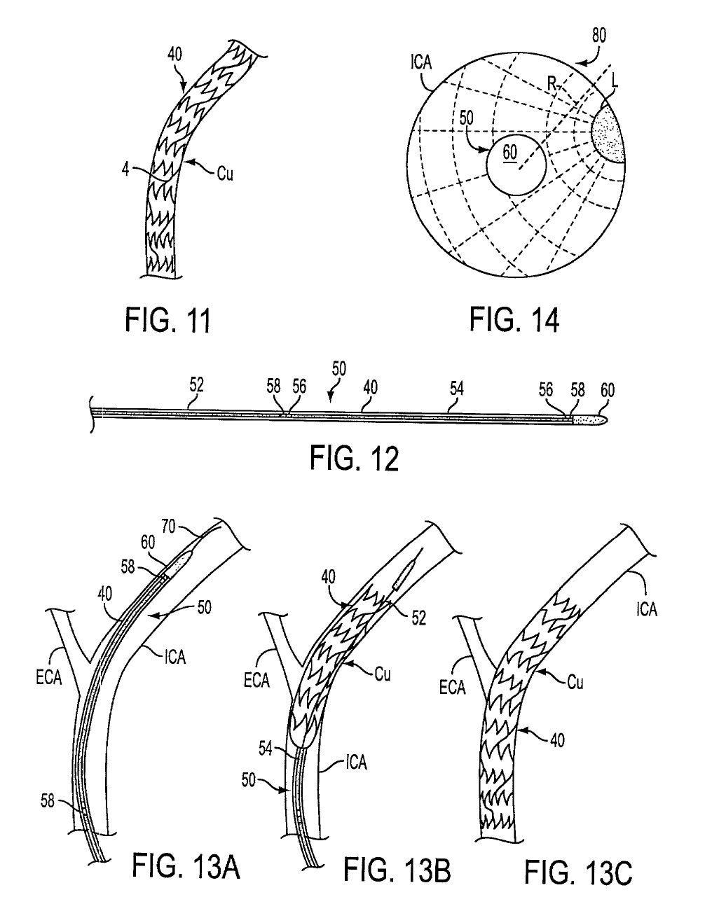

With reference to FIG. 11, an alternative embodiment of stent 1 is described. Prior art stents are commonly formed with substantially straight longitudinal axes. When such a stent is implanted within a tortuous blood vessel, i.e. a blood vessel that does not have a straight longitudinal axis, either the stent or the vessel (or both) deforms to match the profile of the vessel or stent, respectively.

Since previously known self-expanding stents are somewhat flexible, they generally deform at least partially to the curvature of the vessel. However, notably near their ends, these stents also apply localized restoring forces to the wall of the vessel that act to straighten the vessel in the vicinity of the implantation site. As previously known balloon-expandable stents tend to exert higher radial forces, they may apply restoring forces that cause tortuous anatomy to assume the substantially straight profiles of the stents.

For both self-expanding and balloon-expandable embodiments, in circumstances where the vessel wall is thinned or brittle, restoring forces may cause acute puncture or dissection of the vessel, potentially jeopardizing the health of the patient. Alternatively, the restoring forces may cause localized vessel irritation, or may remodel the vessel over time such that it more closely tracks the unstressed, straight profile of the stent. Such remodeling may alter blood flow characteristics through the vessel in unpredictable ways. Restoring forces also may kink the vessel, reducing luminal diameter and blood flow, while increasing blood pressure and turbulence. These and other factors may increase a risk of stenosis or thrombus formation, as well as vessel occlusion.

In FIG. 11, apparatus in accordance with the present invention is provided that is expected to reduce potentially harmful restoring forces applied to tortuous anatomy by prior art stents. Stent 40 comprises curvature Cu in an expanded deployed configuration. Stent 40 also illustratively comprises web structure 4 described hereinabove; however, other structures will be apparent to those of skill in the art. The web structure may be formed, for example, by laser-cutting a tubular member, as discussed previously.

Stent 40 comprising curvature Cu is preferably self-expanding or balloon-expandable. However, Biflex, wire mesh, and other embodiments will be apparent to those of skill in the art, and fall within the scope of the present invention. Self-expanding embodiments of stent 40 are preferably fabricated from a superelastic material, such as a nickel-titanium alloy, e.g. “Nitinol”. Balloon-expandable embodiments may comprise, for example, a stainless steel.

Curvature Cu of stent 40 is configured to match the curvature of an implantation site within a patient's body lumen or body orifice, for example, adapted to match the curvature of a tortuous blood vessel. Thus, when implanted within the vessel, neither the vessel nor the stent need deform to match the other's profile. Curvature matching is thereby expected to reduce localized restoring forces at the implantation site. Curvature may be imparted to stent 40 by a variety of techniques, such as by heat treating the stent while it is arranged with the desired curvature, or by plastically deforming the stent with secondary apparatus, e.g. a curved balloon.

Matching of curvature Cu with the internal profile of a blood vessel or other body lumen may be accomplished by mapping the internal profile of the body lumen, preferably in 3-dimensional space. Then, curvature Cu of stent 40 may be custom-formed accordingly, e.g. by heat treating the stent. Alternatively, secondary apparatus, such as a balloon catheter, may be custom-formed and adapted for plastically deforming stent 40 to impose the curvature. Mapping of the body lumen may be accomplished using a variety of techniques, including ultrasound, e.g. B-mode ultrasound examination, intravascular ultrasound (“IVUS”), angiography, radiography, magnetic resonance imaging (“MRI”), computed tomography (“CT”), and CT angiography.

As an alternative to custom-forming the curvature of stent 40 or the curvature of secondary apparatus for plastically deforming stent 40, a statistical curvature matching technique may be used. Stent 40 or the secondary apparatus may be provided with a standardized curvature Cu that more closely matches an average curvature for a desired body lumen within a specific patient population, as compared to prior art stents. As with custom matching, statistical matching of the curvature may be facilitated or augmented by pre-mapping the intended implantation site.

As a further alternative, stent 40 may be manufactured and stocked in a number of different styles, each having its own predetermined curvature. In this manner, a clinician may select a stent having a degree of curvature most appropriate for the specific anatomy presented by the case at hand.

Beneficially, the present invention provides flexibility in providing stents having a wide variety of curvatures/tortuosities, as needed, as will be apparent to those of skill in the art. Stent 40 is expected to have specific utility at tortuous vessel branchings, for example, within the carotid arteries.

Referring now to FIG. 12, a self-expanding embodiment of stent 40, having pre-imposed curvature in the deployed configuration, is shown in a collapsed delivery configuration within delivery catheter 50. Catheter 50 comprises inner sheath 52 having a guide wire lumen, and outer sheath 54 having a lumen sized for disposal about inner sheath 52. Sheath 52 comprises section 56 of reduced cross section. Stent 40 is collapsed about section 56 of inner sheath 52 between optional radiopaque marker bands 58, such that the stent is flush with the remainder of the inner sheath. Marker bands 58 facilitate longitudinal positioning of stent 40 at an implantation site. Outer sheath 54 is disposed over inner sheath 52 and stent 40, in order to maintain the stent in the collapsed delivery configuration. Sheaths 52 and 54 straighten stent 40 while it is in the delivery configuration, thereby facilitating delivery of the stent to an implantation site.

Delivery catheter 50 optionally may comprise imaging transducer 60 that facilitates radial positioning of stent 40, i.e. that facilitates in vivo radial alignment of curvature Cu of stent 40 with the internal profile of the implantation site. Imaging transducer 60 preferably comprises an IVUS transducer that is coupled to a corresponding imaging system, as described hereinbelow with respect to FIG. 14. An IVUS transducer similar to transducer 60 optionally may also be used to 3-dimensionally map the internal profile of the implantation site prior to advancement of stent 40, thereby allowing custom-manufacture of stent 40.

With reference now to FIGS. 13, a method of using the self-expanding embodiment of stent 40 within tortuous anatomy at a vessel branching is described. In FIGS. 13, stent 40 is illustratively disposed within a patient's carotid arteries, but other implantation sites will be apparent to those of skill in the art. As seen in FIG. 13A, delivery catheter 50, having stent 40 disposed thereon in the collapsed delivery configuration, is advanced over guide wire 70 to an implantation site within internal carotid artery ICA that spans the branching of external carotid artery ECA. The implantation site may comprise a stenosed or otherwise damaged portion of the artery.

Stent 40 has a curvature Cu in the expanded deployed configuration of FIG. 11 that tracks the internal profile of internal carotid artery ICA at the implantation site. As discussed previously, curvature Cu may be custom-formed, statistically chosen, or selected from a number of pre-manufactured shapes to better track the curvature of the artery. Such selection may be facilitated or augmented by mapping the profile of the ICA, using techniques described hereinabove.

In order to properly align curvature Cu of stent 40 with the internal profile of the implantation site within internal carotid artery ICA, optional radiopaque marker bands 58 and optional imaging transducer 60 of delivery catheter 50 may respectively be used to longitudinally and radially position stent 40 at the implantation site. Longitudinal positioning of stent 40 may be accomplished by imaging radiopaque marker bands 58, e.g. with a fluoroscope. The implantation site is then positioned between the marker bands, thereby longitudinally orienting stent 40.

Referring to FIG. 14, in conjunction with FIGS. 13, a technique for radial positioning is described. Imaging transducer 60 preferably comprises an IVUS transducer. Transducer 60 may be either a forward-looking IVUS transducer, or a standard radial-looking IVUS transducer. FIG. 14 provides illustrative IVUS image 80, collected from transducer 60.

In FIG. 14, when using a forward-looking IVUS transducer 60, lumen L of internal carotid artery ICA can be seen curving away from the longitudinal axis of transducer 60 of delivery catheter 50. Reference line R has been superimposed on image 80 and corresponds to the axis of curvature of stent 40. Thus, rotation of catheter 50, and thereby transducer 60 and stent 40, causes rotation of reference line R within image 80. In order to radially orient stent 40 with respect to the implantation site, reference line R is aligned with lumen L.

Referring still to FIG. 14, when using a standard radial-looking IVUS transducer 60, side-branching external carotid artery ECA may be imaged. By comparing the position of the external carotid in the IVUS image of FIG. 14 to its position in the fluoroscopic images of FIGS. 13, catheter 50 may be rotated to radially align reference line R relative to the position of external carotid artery ECA in FIGS. 13, thereby radially aligning curvature Cu of stent 40 with the curvature of internal carotid artery ICA.

As an alternative technique, both longitudinal and radial positioning of stent 40 may be performed with transducer 60. This is accomplished by creating a 3-dimensional map of the implantation site with transducer 60, by collecting and stacking a series of cross-sectional IVUS images taken along the length of the implantation site. Stent 40 is then positioned with respect to this map. If the vessel was mapped prior to delivery of catheter 50 and stent 40, longitudinal positioning may be accomplished by referencing IVUS image 80 with the previously-conducted mapping, and by advancing catheter 50 until image 80 matches the cross-section of the previous mapping at the proper location.

As yet another technique, both longitudinal and radial positioning of stent 40 may be achieved with radiopaque marker bands 58. Longitudinal positioning may be achieved as described previously, while radial positioning may be achieved by varying the radiopacity of the bands about their circumference, such that the bands comprise a visually recognizable alteration in radiopacity along the axis of curvature of stent 40. This alteration in radiopacity is aligned with the axis of curvature of the implantation site.

Referring back now to FIGS. 13, in FIG. 13B, once stent 40 has been radially and longitudinally oriented with respect to internal carotid artery ICA, outer sheath 54 of delivery catheter 50 is gradually withdrawn with respect to inner sheath 52. Stent 40 self-expands to the deployed configuration, and delivery catheter 50 and guide wire 70 are removed from the artery, as in FIG. 13C. Curvature Cu of stent 40 tracks the internal profile of internal carotid artery ICA, thereby reducing restoring forces applied to the vessel.

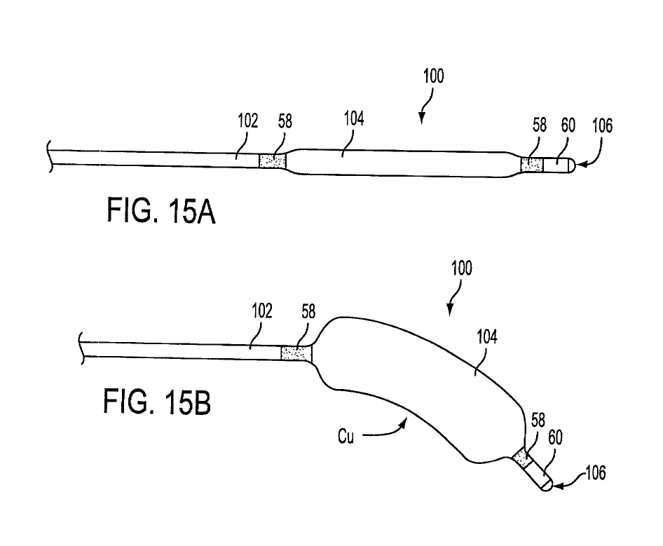

With reference to FIGS. 15, secondary apparatus in accordance with the present invention for applying curvature to a balloon-expandable embodiment of stent 40 is described. Secondary apparatus 100 comprises balloon catheter 102 having balloon 104. Secondary apparatus 102 also preferably comprises guide wire lumen 106, as well as radiopaque marker bands 58 and imaging transducer 60, as described hereinabove with respect to FIGS. 13 and 14. Balloon 104, and by extension secondary apparatus 100, is substantially straight in the collapsed delivery configuration of FIG. 15A, but comprises curvature Cu in the expanded deployed configuration of FIG. 15B.

Curvature Cu may be applied to balloon 104 using techniques described hereinabove. For example, balloon 104 may be heat-treated while the balloon is arranged with the desired curvature. Heat treating of balloon 104 may be accomplished while the balloon is in either the delivery or deployed configuration, or while the balloon is in an intermediary configuration. Additionally, curvature Cu of balloon 104 may be matched to the internal profile of a treatment site using, for example, custom-matching or statistical-matching techniques, as described previously.

Embodiments of stent 40 for use with the apparatus of FIGS. 15 are preferably manufactured without curvature Cu, and may comprise, for example, stent 1 of FIGS. 1-10. As will be clear to those of skill in the art, a balloon-expandable embodiment of stent 40 may be crimped onto balloon 104 while the balloon is in the collapsed delivery configuration. When the balloon is expanded to the deployed configuration at a tortuous treatment site within a patient, curvature Cu of balloon 104 plastically deforms stent 40 and imposes curvature Cu on the stent. Alignment of curvature Cu with the curvature of the tortuous anatomy may be accomplished using, for example, techniques described hereinabove with respect to FIGS. 13 and 14. Thus, a method for placing profile-matched balloon-expandable stents in tortuous anatomy is clear to those of skill in the art from FIGS. 10 in conjunction with FIGS. 13 and 14.

Although preferred illustrative embodiments of the present invention are described hereinabove, it will be evident to one skilled in the art that various changes and modifications may be made therein without departing from the invention. For example, stent 40 may further comprise coating C, described hereinabove. Additionally, alternative embodiments of secondary apparatus 100 for plastically deforming stent 40, which do not comprise balloons, may be provided. It is intended in the appended claims to cover all such changes and modifications that fall within the true spirit and scope of the invention.

What is claimed is:

1. A stent adapted for expansion from a collapsed delivery configuration to an expanded deployed configuration, the stent having, in the deployed configuration, a curvature relative to a longitudinal axis of the stent.

2. The stent of claim 1 further comprising a self-expandable structure adapted for expansion from the collapsed delivery configuration to the expanded deployed configuration.

3. The stent of claim 2, wherein the self-expandable structure of the stent is formed by laser-cutting a tubular member.

4. The stent of claim 1, wherein the curvature of the stent is configured to match an internal profile of an implantation site within a patient's body lumen.

5. The stent of claim 4, wherein the curvature of the stent is configured to reduce restoring forces applied by the stent to the implantation site.

6. The stent of claim 4, wherein the curvature of the stent is configured to match a 3-dimensional map of the internal profile of the implantation site.

7. The stent of claim 4, wherein the curvature of the stent is custom-manufactured to match the internal profile of the implantation site.

8. The stent of claim 4, wherein the curvature of the stent is statistically matched to the internal profile of the implantation site.

9. The stent of claim 1, wherein the curvature of the stent is formed by heat treating the stent while it is arranged with the desired curvature.

10. The stent of claim 6, wherein the 3-dimensional map is formed by a technique chosen from the group consisting of ultrasound imaging, intravascular ultrasound imaging, angiography, radiography, magnetic resonance imaging, computed tomography, and computed tomography angiography.

11. The stent of claim 1 further comprising a delivery catheter adapted to selectively maintain the stent in the collapsed delivery configuration.

12. The stent of claim 11, wherein the delivery catheter comprises an inner sheath and an outer sheath, the outer sheath removably disposed about the inner sheath, the stent concentrically disposed between the inner and outer sheaths in the collapsed delivery configuration.

13. The stent of claim 12, wherein the delivery catheter further comprises radiopaque marker bands, the stent disposed between the marker bands.

14. The stent of claim 12, wherein the delivery catheter further comprises an imaging transducer.

15. The stent of claim 1, wherein the stent is fabricated from a material chosen from the group consisting of superelastic materials, biocompatible materials, and biodegradable materials.

16. The stent of claim 1, wherein the stent is flexible in the collapsed delivery configuration.

17. The stent of claim 1, wherein a thickness of a wall of the stent changes along the longitudinal axis of the stent.

18. The stent of claim 1 further comprising a coating at least partially covering the stent.

19. The stent of claim 18 wherein the coating is configured to perform an action chosen from the group consisting of retarding restenosis, retarding thrombus formation, and delivering therapeutic agents to the patient's blood stream.

20. The stent of claim 1 further comprising:
a tubular body with a wall having a web structure,
the web structure comprising a plurality of interconnected, neighboring web patterns, each web pattern having a plurality of adjoining webs, each adjoining web comprising a central section interposed between first and second lateral sections,
wherein the central section is substantially parallel to a longitudinal axis of the stent when in the collapsed delivery configuration, each of the first lateral sections joins the central section at a first angle, each of the second lateral sections joins the central section at a second angle, and adjacent ones of the neighboring web patterns have alternating concavity.

21. The stent of claim 20, wherein the first angle comprises a first obtuse angle, and wherein the second angle comprises a second obtuse angle.

22. The stent of claim 20, wherein each adjoining web has a bowl-like appearance.

23. The stent of claim 20 further comprising a plurality of connection elements configured to interconnect the plurality of web patterns.

24. The stent of claim 20 further comprising a plurality of transition sections configured to interconnect neighboring web patterns.

25. The stent of claim 20, wherein the number of adjoining webs that span a circumference of the stent is selected corresponding to a vessel diameter in which the stent is to be implanted.

26. The stent of claim 1 further comprising secondary apparatus for plastically deforming the stent during expansion of the stent from the collapsed delivery configuration to the expanded deployed configuration, thereby imposing the curvature along the longitudinal axis of the stent in the deployed configuration.

27. The stent of claim 26, wherein the secondary apparatus comprises a balloon catheter adapted for expansion from a collapsed delivery configuration to an expanded deployed configuration, the balloon catheter comprising curvature along a longitudinal axis of the catheter in the deployed configuration.

28. The stent of claim 27, wherein the curvature of the balloon catheter is configured to match an internal profile of an implantation site within a patient's body lumen.

29. The stent of claim 28, wherein the curvature of the balloon catheter is configured to match a 3-dimensional map of the internal profile of the implantation site.

30. The stent of claim 28, wherein the curvature of the balloon catheter is custom-manufactured or is statistically matched to the internal profile of the implantation site.

31. The stent of claim 27, wherein the curvature of the balloon catheter is formed by heat treating the balloon catheter while it is arranged with the desired curvature.

32. A method for stenting at a tortuous implantation site within a patient's vessel with reduced restoring forces, the method comprising:
providing a stent adapted for expansion from a collapsed delivery configuration to an expanded deployed configuration, the stent having, in the deployed configuration, a curvature relative to a longitudinal axis of the stent;
percutaneously delivering the stent to the tortuous implantation site within the patient's vessel in the collapsed delivery configuration;
aligning the stent with an internal profile of the implantation site; and
deploying the stent to the expanded deployed configuration, the stent engaging the implantation site and the curvature of the stent tracking the site's internal profile.

33. The method of claim 32, wherein providing the stent further comprises providing the stent with a self-expandable structure adapted for expansion from the collapsed delivery configuration to the expanded deployed configuration.

34. The method of claim 32, wherein providing the stent further comprises providing the stent with a tubular body having a wall with a web structure,
the web structure comprising a plurality of interconnected, neighboring web patterns, each web pattern having a plurality of adjoining webs, each adjoining web comprising a central section interposed between two lateral sections, wherein the central section is substantially parallel to a longitudinal axis of the stent when in the collapsed delivery configuration, and adjacent ones of the neighboring web patterns have alternating concavity.

35. The method of claim 32 wherein deploying the stent to the expanded configuration comprises releasing the stent from a mechanical restraint.

36. The method of claim 32, wherein aligning the stent with an internal profile of the implantation site comprises radially and longitudinally aligning the curvature of the stent with the internal profile.

37. The method of claim 32, wherein aligning the stent further comprises using an imaging modality to align the curvature of the stent.

38. The method of claim 32, wherein providing a stent comprising curvature further comprises matching the curvature with the internal profile of the implantation site.

39. The method of claim 38, wherein matching the curvature with the internal profile of the implantation site comprises custom-matching or statistically-matching the curvature.

40. The method of claim 38, wherein matching the curvature with the internal profile comprises plastically deforming the stent with secondary apparatus having curvature that matches the curvature of the internal profile.

41. The method of claim 40, wherein the secondary apparatus comprises a balloon catheter.

42. Apparatus for plastically deforming a stent, the apparatus comprising a balloon catheter adapted for expansion from a collapsed delivery configuration to an expanded deployed configuration, the balloon catheter comprising curvature along a longitudinal axis of the catheter in the deployed configuration.

43. The apparatus of claim 42, wherein the curvature of the balloon catheter is configured to match an internal profile of an implantation site within a patient's body lumen.

44. The apparatus of claim 42, wherein the curvature of the balloon catheter is configured to match a 3-dimensional map of the internal profile of the implantation site.

45. The apparatus of claim 42, wherein the curvature of the balloon catheter is custom-manufactured or is statistically matched to the internal profile of the implantation site.

46. The apparatus of claim 42, wherein the curvature of the balloon catheter is formed by heat treating the balloon catheter while it is arranged with the desired curvature.

47. The apparatus of claim 42 further comprising a stent disposed about the balloon catheter.
